(19) United States
(12) Patent Application Publication
(10) Pub. No.: US 2008/0293638 A1
(43) Pub. Date: Nov. 27, 2008

(54) ALBUMIN-FUSED CILIARY NEUROTROPHIC FACTOR

(75) Inventors: Mathias Jurs, Waldems Reichenbach (DE); Thomas Weimer, Gladenbach (DE); Hans-Peter Hauser, Marburg (DE); Darrell Sleep, West Bridgford (GB)

Correspondence Address: FOX ROTHSCHILD LLP, 1101 MARKET STREET, SUITE 2600, PHILADELPHIA, PA, 19107-2950, US

(73) Assignee: NOVOZYMES BIOPHARMA UK LIMITED, Nottingham (GB)

(21) Appl. No.: 12/167,537

(22) Filed: Jul. 3, 2008

Related U.S. Application Data
(63) Continuation of application No. 11/051,615, filed on Feb. 4, 2005, now abandoned.

(51) Int. Cl. A61K 38/38; C07K 14/76

(52) U.S. Cl. 514/12; 530/362

(57) ABSTRACT

The invention relates to a fusion protein comprising an albumin, or a fragment or a variant or a derivative thereof and at least one biologically active peptide which activates the ciliary neurotrophic factor (CNTF) receptor, or a fragment or variant or a derivative thereof.

### FIELD OF THE INVENTION

The invention relates to a fusion protein comprising an albumin, or a fragment or a variant or a derivative thereof, and at least one biologically active peptide or protein which activates the ciliary neurotrophic factor (CNTF) receptor, or a fragment or variant or a derivative thereof.

### BACKGROUND OF THE INVENTION

Regulation of daily energy homeostasis stands mainly under the central control of a few discrete nuclei [1] in the basal hypothalamus (ventromedial nucleus, dorsomedial nucleus, paraventricular nucleus, and lateral hypothalamus), but there are also other central nervous structures (cerebral cortex, limbic region, brainstem, pituitary gland, autonomic preganglionic neurons, dorsal vagal complex) as well as peripheral nervous structures (sympathetic preganglionic neurons) involved [2].

Beside the central and peripheral nervous regulation, peripheral organs involved in the balance of energy homeostasis are the gastrointestinal tract (stomach, gut), the pancreas, the adipose tissue, the muscle tissue, the adrenal glands and the thyroid gland.

The process of regulation is complex and peripheral organs such as the gastrointestinal tract can release hormones after food intake (e.g. CCK (cholecystokinin)), which cause a decrease of appetite-increasing hormones in the hypothalamus. Furthermore, leptin, released by fat tissue after food intake, has a negative regulatory effect on e.g. NPY (Neuropeptide Y) which is one of the major centrally active appetite-inducing hormones. Centrally released hormones, on the other side, may have a peripheral effect as well (e.g. β₃-adrenergic agonists, uncoupling protein (UCPs)) increasing thermogenesis. The interested reader is referred to actual reviews covering the whole spectrum [1-6].

AXOKINE® (Regeneron, Inc, Tarrytown, N.Y., USA) is a mutant version of the CNTF. AXOKINE® is the truncated form of CNTF where the last 15 C-terminal amino acids have been removed. To enhance the stability of the molecule, glutamine is replaced by arginine at position 63 and the free cysteine at position 17 is replaced by alanine [7].

The weight-reducing effect of CNTF was discovered by chance during clinical trials in subjects suffering from motor-neurone disease [8]. Further studies revealed that the mechanism of action provided by CNTF to induce loss of weight is similar to leptin with the difference that CNTF is also active in diet-induced obesity [7]. Studies in animals using AXOKINE® confirmed the weight-loss inducing capacity by this CNTF-mutant similar to the CNTF-mechanism.

CNTF has a negative regulatory effect on the synthesis of NPY, Agouti-related peptide (AGRP) and gamma-aminobutyric acid (GABA), all known to stimulate feeding.

CNTF was shown to cross the blood brain barrier (BBB) in an intact form [10]. Recently it was shown that CNTF is transported via a saturable transport system with a rate of entry Kᵢ of 4.60 (±0.78)×10⁻⁴ mL/g min [11].

The BBB is a highly regulated barrier to molecules from the blood preventing them to enter the brain tissue [13]. It is formed by brain capillary endothelial cells.

From Lambert et al. [7] we know that AXOKINE® worked in leptin deficient (ob/ob) and wild-type (diet-induced obesity, DIO) mice. The most effective dose was 300 μg/kg b.w. of AXOKINE®, but effects were also observed with 100 μg/kg b.w. Weight loss achieved was mainly due to loss of fat tissue, avoiding loss of lean body mass.

Furthermore, there was no rebound effect in mice treated with AXOKINE® whereas mice not treated with AXOKINE® and receiving the diet the AXOKINE® treated animals consumed (pair fed group), quickly regained their original weight.

Phase I data were published by Guler et al. in the International Journal of Obesity [14]. AXOKINE® was tolerated well, no subjects dropped out and the majority of all adverse events (AE) were considered to be “mild”. Dose limiting toxicities were vomiting and nausea in part A at 16 μg/kg b.w. Injection site reactions were the most frequently reported AE in the drug treated subjects, followed by decreased appetite, nausea, headache, and diarrhea. Herpetiform mouth lesions were noted in some subjects.

One subject suffered a transient Bell's palsy (palsy of the VIIth cranial nerve, the facial nerve, where the mimic muscles of the face get paralysed) 10 days after the end of treatment with AXOKINE® at 1 μg/kg b.w./day. At the higher doses, increased C-reactive protein and erythrocyte sedimentation rate (ESR), and decreased serum Fe⁺ were noted. In a dose-dependent fashion, heart rate increased and body temperature tended to be higher.

A multicenter, randomised, double-blind, placebo-controlled, dose-ranging phase II study [15] involving 170 severely or morbidly obese patients has evaluated that patients receiving the optimal dose of AXOKINE® (1.0 μg/kg) over the 12-week treatment period averaged a 10-pound greater [16] weight loss than placebo recipients (p<0.001).

Weight loss was maintained for 4 months after the last administration of AXOKINE® in patients from the 8-week treatment group [17, 18]. No serious adverse events were reported. The most frequently reported adverse event was dose-dependent, mild injection site reaction (site redness) that occurred in all patients, including placebo group. The administration of AXOKINE® was associated with cough and nausea, which occurred most frequently after the 2.0 μg/kg b.w. dose of the agent. No increase in herpes simplex virus infections was observed in AXOKINE® recipients compared with placebo. Comparable proportions of AXOKINE®, and (58-74%), and placebo (61%), recipients completed the full 12-week study.

In a phase III placebo-controlled study 1467 AXOKINE-treated subjects and 501 placebo-treated subjects demonstrated that:
AXOKINE® treatment, when compared with placebo, achieved statistical significance with regard to both primary endpoints of the study:
   A greater proportion of AXOKINE®-treated patients lost at least 5% of their initial body weight compared with placebo-treated patients (25.1% vs. 17.6%, p<0.001)
   Participants receiving AXOKINE® experienced a greater average weight loss than those receiving placebo (6.2 lbs vs. 2.6 lbs, p<0.001)
AXOKINE® treatment achieved statistically significant results in two of the three secondary endpoints, such as proportion of subjects losing at least 10% of their initial body weight (11.3% vs. 4.2%, p<0.001)
AXOKINE® treatment was generally well-tolerated. Adverse events were generally characterized as mild to moderate and no pattern of serious or severe adverse events emerged. The most notable adverse effects as compared with placebo were injection site reactions, nausea and cough, which were largely characterized as mild
AXOKINE®-associated weight loss was limited by the development of antibodies beginning after about three months of AXOKINE® treatment. However, more than 30% of the total 1467 subjects treated with AXOKINE® did not develop antibodies by the end of one year

### SUMMARY OF THE INVENTION

In one aspect of the invention, the invention is a fusion protein comprising an albumin, in particular a human serum albumin, or a fragment or a variant or a derivative thereof, which has an albumin activity, and at least one biologically active peptide or protein which activates the ciliary neurotrophic factor (CNTF) receptor, or a fragment or variant or a derivative thereof.

In different embodiments, CNTF or albumin may be a fragment or a derivative, or both as in the case of AXOKINE®, or a variant. The albumin fusion protein may be a therapeutic agent.

In another aspect, the invention is a method for extending the half-life of CNTF in a mammal. The method entails linking a CNTF to an albumin to form an albumin-fused CNTF and administering the albumin-fused CNTF to a mammal. Typically, the half-life of the albumin-fused CNTF is extended by at least 2-fold to at least 50-fold over the half-life of the CNTF lacking the linked albumin.

By using either the transport system for CNTF or unspecific transport systems across the blood brain barrier (BBB) like e.g. transcytosis, the intracerebral concentration of albumin fused AXOKINE® is expected to be increased. Due to the increased plasma concentration of the albumin-fused AXOKINE® over time at the BBB compared to the non-fused AXOKINE® a higher influx of albumin-fused AXOKINE® will take place via transcytosis.

Further, the invention entails a method for treating obesity in a mammal. The method comprises linking CNTF to an albumin to form an albumin-fused CNTF and administering the albumin-fused CNTF to the mammal. The invention also encompasses a method for potentially minimizing side effects (e.g. nausea, headache) associated with the treatment of a mammal with CNTF in moderately higher concentrations. The method comprises linking said CNTF to an albumin to form an albumin-fused CNTF and administering said albumin-fused CNTF to said mammal.

### BRIEF DESCRIPTION OF THE DRAWINGS

FIG. 1. Pharmacokinetics of non-fused AXOKINE® in rabbits (i.v.)

FIG. 2. Pharmacokinetics of C- and N-terminal fused AXOKINE® in rabbits (i.v.)

FIG. 3. Pharmacokinetics of C- and N-terminal fused AXOKINE® in rabbits (s.c.)

FIG. 4. Weight loss curve of leptin deficient mice treated with non-fused AXOKINE®

FIG. 5. Weight loss curve of leptin deficient mice treated with C-terminal fused AXOKINE®

FIG. 6. Weight loss curve of wild-type mice treated with non-fused AXOKINE®

FIG. 7. Weight loss curve of wild-type mice treated with C-terminal fused AXOKINE®

FIG. 8. Amino acid sequence of the mature C-terminal AXOKINE® (Seq. ID: 1)

FIG. 9. Amino acid sequence of the mature C-terminal rHA-3×FLAG-(cleavable) AXOKINE® (Seq. ID: 2)

FIG. 10. Amino acid sequence of the mature N-terminal AXOKINE® (Seq. ID: 3)

FIG. 11. Map of the C-terminal fused AXOKINE®

FIG. 12. Map of the C-terminal rHA-3xFLAG-(cleavable) AXOKINE®

FIG. 13. Map of the N-terminal fused AXOKINE®

FIG. 14. Weight loss curve of leptin deficient mice treated every third day with non-fused AXOKINE® and C-terminal fused AXOKINE®

FIG. 15. Weight loss curve of leptin deficient mice treated daily with non-fused AXOKINE® and C-terminal fused AXOKINE®

### DETAILED DESCRIPTION OF THE INVENTION

### Definitions

Ciliary neurotrophic factor (CNTF) means any molecule which is an analogue, homologue, fragment, or a derivative of naturally occurring CNTF which possesses a single biological activity of the naturally occurring CNTF. A preferred CNTF is AXOKINE®. Another CNTF mutant (Ser166Asp/Gln167H is) has been described in the PCT Application WO 98/22128, which, from position 159 to position 178, has the following amino acid sequence:

(159-178; Seq. ID: 4)

Leu Lys Val Leu Gln Glu Leu Asp His Trp Thr Val
Arg Ser Ile His Asp Leu Arg Phe

AXOKINE® is a mutant version of the CNTF. AXOKINE® is the truncated form of CNTF where the last 15 c-terminal amino acids have been removed. To enhance the stability of the molecule, glutamine is replaced by arginine at position 63 and the free cysteine at position 17 is replaced by alanine [7]

N-terminal-AXOKINE® is a fusion of the C-terminal end of AXOKINE® to the N-terminal end of human serum albumin as described in example 1.

C-terminal-AXOKINE® is a fusion of the N-terminal end of AXOKINE® to the C-terminal end of human serum albumin as described in example 1.

Cleavable AXOKINE® as described in example 1 is a C-terminal fusion of AXOKINE® to human serum albumin which has an enterokinase cleavage site between the CNTF portion and albumin which was used to generate cleaved or non-fused AXOKINE® which was used as a control to the N- and C-terminal fusions.

Albumin

The terms human serum albumin (HSA) and human albumin (HA) are used interchangeably herein. The terms “albumin” and “serum albumin” are broader, and encompass human serum albumin (and fragments and variants thereof) as well as albumin from other species (and fragments and variants thereof).

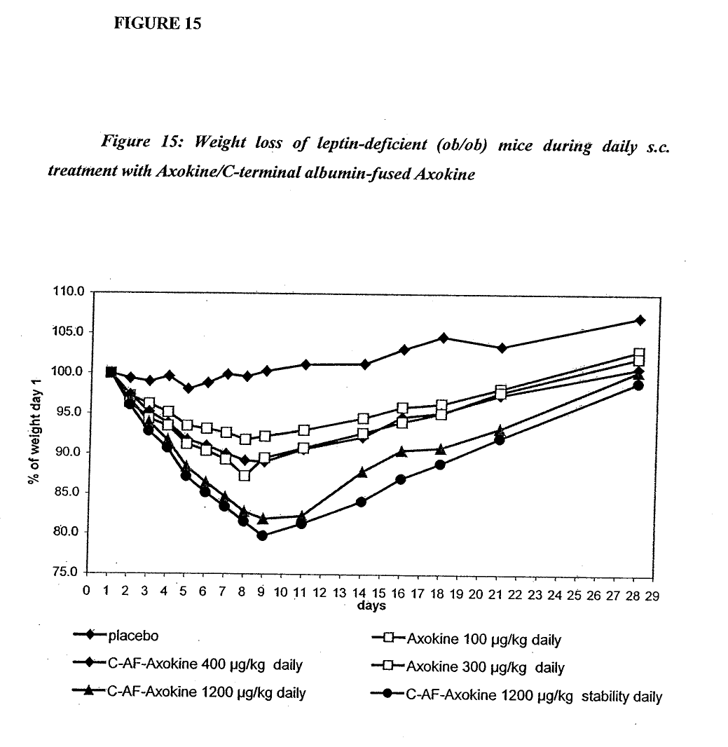

As used herein, “albumin” refers collectively to albumin protein or amino acid sequence, or an albumin fragment or variant, having one or more functional activities (e.g., biological activities) of albumin. In particular, “albumin” refers to human albumin or fragments thereof (see EP 201 239, EP 322 094, WO 97/24445, WO95/23857) especially the mature form of human albumin as shown in FIG. 15 (SEQ ID NO:18) of WO 01/79480, hereby incorporated in its entirety by reference herein, or albumin from other vertebrates or fragments thereof, or analogs or variants of these molecules or fragments thereof.

This sequence of FIG. 15 of WO 01/79480 is in this application referred to as the “WO '480 sequence”.

The human serum albumin protein used in the albumin fusion proteins in the examples contains one or both of the following sets of point mutations with reference to WO '480 SEQUENCE: Leu-407 to Ala, Leu-408 to Val, Val-409 to Ala, and Arg-410 to Ala; or Arg-410 to Ala, Lys-413 to Gln, and Lys-414 to Gln (see, e.g., International Publication No. WO95/23857, hereby incorporated in its entirety by reference herein). In other embodiments, albumin fusion proteins of the invention that contain one or both of above-described sets of point mutations have improved stability/resistance to yeast Yap3p proteolytic cleavage, allowing increased production of recombinant albumin fusion proteins expressed in yeast host cells.

As used herein, a portion of albumin sufficient to prolong or extend the in vivo half-life, therapeutic activity, or shelf-life of the CNTF refers to a portion of albumin sufficient in length or structure to stabilize, prolong or extend the in vivo half-life, therapeutic activity or shelf-life of the CNTF portion of the albumin fusion protein compared to the in vivo half-life, therapeutic activity, or shelf-life of the CNTF in the non-fusion state. The albumin portion of the albumin fusion proteins may comprise the full length of the HA sequence as described above, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA.

The albumin portion of the albumin fusion proteins of the invention may be a variant of normal HA. The CNTF portion of the albumin fusion proteins of the invention may also be variants of nature-identical CNTF. The term “variants” includes insertions, deletions and substitutions, either conservative or non conservative, where such changes do not substantially alter one or more of the oncotic, useful ligand-binding and non-immunogenic properties of albumin, or the active site, or active domain which confers the therapeutic activities of the CNTF.

In particular, the albumin fusion proteins of the invention may include naturally occurring polymorphic variants of human albumin and fragments of human albumin, for example those fragments disclosed in EP 322 094 (namely HA (Pn), where n is 369 to 419). The albumin may be derived from any vertebrate, especially any mammal, for example human, cow, sheep, or pig. Non-mammalian albumins include, but are not limited to, hen and salmon. The albumin portion of the albumin fusion protein may be from a different animal than the CNTF portion.

Generally speaking, an HA fragment or variant will be at least 100 amino acids long, optionally at least 150, 200, 300, 400, 500, 550, 570 or 580 amino acids long. The HA variant may consist of or alternatively comprise at least one whole domain of HA, for example domains 1 (amino acids 1-194 of WO '480 SEQUENCE), 2 (amino acids 195-387 of WO '480 SEQUENCE), 3 (amino acids 388-585 of WO '480 SEQUENCE), I+2 (1-387 of WO '480 SEQUENCE), 2+3 (195-585 of WO '480 SEQUENCE) or 1+3 (amino acids 1-194 of WO '480 SEQUENCE+amino acids 388-585 of WO '480 SEQUENCE). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val 315 and Glu492 to Ala511.

The albumin portion of an albumin fusion protein of the invention may comprise at least one subdomain or domain of HA or conservative modifications thereof. If the fusion is based on subdomains, some or all of the adjacent linker is may optionally be used to link to the CNTF moiety.

An albumin “variant” may comprise, or alternatively consist of, an amino acid sequence which is at least 80%, 85%, 90%, 95%, 96%, 97%, 98%, 99% or 100%, identical to the amino acid sequence of albumin as shown in FIG. 15 (SEQ ID NO:18) of WO 01/79480. Further polypeptides encompassed by the invention are polypeptides encoded by polynucleotides which hybridize to the complement of a nucleic acid molecule encoding an amino acid sequence of the invention under stringent hybridization conditions (e.g., hybridization to filter bound DNA in 6× Sodium chloride/Sodium citrate (SSC) at about 45° C., followed by one or more washes in 0.2×SSC, 0.1% SDS at about 50-65° C.), under highly stringent conditions (e.g., hybridization to filter bound DNA in 6× sodium chloride/Sodium citrate (SSC) at about 45° C., followed by one or more washes in 0.1×SSC, 0.2% SDS at about 68° C.), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, the amino acid sequence of an albumin fusion protein of the invention or a fragment thereof (such as the CNTF portion of the albumin fusion protein or the albumin portion of the albumin fusion protein), can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci.6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size-sequence length, Gap Penalty=5, Gap Size Penalty-0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The variant will usually have at least 75% (preferably at least about 80%, 90%, 95% or 99%) sequence identity with a length of normal HA or CNTF which is the same length as the variant. Homology or identity at the nucleotide or amino acid sequence level is determined by BLAST (Basic Local Alignment Search Tool) analysis using the algorithm employed by the programs blastp, blastn, blastx, tblastn and tblastx (Karlin et al., Proc. Natl. Acad. Sci. USA 87: 2264-2268 (1990) and Altschul, J. Mol. Evol. 36: 290-300 (1993), fully incorporated by reference) which are tailored for sequence similarity searching.

The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters may be adjusted as follows: Q=10 (gap creation penalty); R=10 (gap extension penalty); wink=1 (generates word hits at every wink h position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The polynucleotide variants of the invention may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, polypeptide variants in which less than 50, less than 40, less than 30, less than 20, less than 10, or 5-50, 5-25, 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host, such as, yeast or E. coli).

Albumin Fusion Proteins

The present invention relates generally to albumin fusion proteins and methods of treating, preventing, or ameliorating diseases or disorders. As used herein, “albumin fusion protein” refers to a protein formed by the fusion of at least one molecule of albumin (or a fragment or variant thereof) to at least one molecule of a CNTF (or fragment or variant thereof). An albumin fusion protein of the invention comprises at least a fragment or variant of a CNTF and at least a fragment or variant of human serum albumin, which are associated with one another, such as by genetic fusion (i.e., the albumin fusion protein is generated by translation of a nucleic acid in which a polynucleotide encoding all or a portion of a CNTF is joined in-frame with a polynucleotide encoding all or a portion of albumin) to one another. The CNTF and albumin protein, once part of the albumin fusion protein, may be referred to as a “portion”, “region” or “moiety” of the albumin fusion protein.

In one embodiment, the invention provides an albumin fusion protein comprising, or alternatively consisting of, a CNTF and a serum albumin protein. In other embodiments, the invention provides an albumin fusion protein comprising, or alternatively consisting of, a biologically active and/or therapeutically active fragment of a CNTF and a serum albumin protein. In other embodiments, the invention provides an albumin fusion protein comprising, or alternatively consisting of, a biologically active and/or therapeutically active variant of a CNTF and a serum albumin protein. In further embodiments, the serum albumin protein component of the albumin fusion protein is the mature portion of serum albumin.

In further embodiments, the invention provides an albumin fusion protein comprising, or alternatively consisting of CNTF and a biologically active and/or therapeutically active fragment of serum albumin. In further embodiments, the invention provides an albumin fusion protein comprising, or alternatively consisting of, a CNTF and a biologically active and/or therapeutically active variant of serum albumin. In some embodiments, the CNTF portion of the albumin fusion protein is the mature portion of the CNTF.

In further embodiments, the invention provides an albumin fusion protein comprising, or alternatively consisting of, a biologically active and/or therapeutically active fragment or variant of a CNTF and a biologically active and/or therapeutically active fragment or variant of serum albumin. In some embodiments, the invention provides an albumin fusion protein comprising, or alternatively consisting of, the mature portion of a CNTF and the mature portion of serum albumin.

In one embodiment, the albumin fusion protein comprises HA as the N-terminal portion, and a CNTF as the C-terminal portion. Alternatively, an albumin fusion protein comprising HA as the C-terminal portion, and a CNTF as the N-terminal portion may also be used.

In other embodiments, the albumin fusion protein has a CNTF fused to both the N-terminus and the C-terminus of albumin. In one embodiment, the CNTF proteins fused at the N- and C-termini are the same CNTF proteins. In another embodiment, the CNTF proteins fused at the N- and C-termini are different CNTF proteins. In another embodiment, the CNTF proteins fused at the N- and C-termini are different CNTF proteins which may be used to treat or prevent the same disease, disorder, or condition. In another embodiment, the CNTF proteins fused at the N- and C-termini are different CNTF proteins which may be used to treat or prevent diseases or disorders which are known in the art to commonly occur in patients simultaneously.

In addition to albumin fusion protein in which the albumin portion is fused N-terminal and/or C-terminal of the CNTF portion, albumin fusion proteins of the invention may also be produced by inserting the CNTF or peptide of interest into an internal region of HA. For instance, within the protein sequence of the HA molecule a number of loops or turns exist between the end and beginning of α-helices, which are stabilized by disulphide bonds. The loops, as determined from the crystal structure of HA (PDB identifiers 1AO6, 1BJ5, 1BKE, 1BM0, 1E7E to 1E7I and 1UOR) for the most part extend away from the body of the molecule. These loops are useful for the insertion, or internal fusion, of therapeutically active peptides, particularly those requiring a secondary structure to be functional, to essentially generate an albumin molecule with specific biological activity.

Loops in human albumin structure into which peptides or polypeptides may be inserted to generate albumin fusion proteins of the invention include: Va154-Asn61, Thr76-Asp89, Ala92-Glu100, Gln170-Ala176, His247-Glu252, Glu266-Glu277, Glu280-His288, Ala362-Glu368, Lys439-Pro447, Val462-Lys475, Thr478-Pro486, and Lys560-Thr566. In other embodiments, peptides or polypeptides are inserted into the Val54-Asn61, Gln170-Ala176, and/or Lys560-Thr566 loops of mature human albumin (WO '480 SEQUENCE).

Peptides to be inserted may be derived from either phage display or synthetic peptide libraries screened for specific biological activity or from the active portions of a molecule with the desired function. Additionally, random peptide libraries may be generated within particular loops or by insertions of randomized peptides into particular loops of the HA molecule and in which all possible combinations of amino acids are represented.

Such library(s) could be generated on HA or domain fragments of HA by one of the following methods:

(a) randomized mutation of amino acids within one or more peptide loops of HA or HA domain fragments. Either one, more or all the residues within a loop could be mutated in this manner;

(b) replacement of, or insertion into one or more loops of HA or HA domain fragments (i.e., internal fusion) of a randomized peptide(s) of length Xₙ (where X is an amino acid and n is the number of residues;

(c) N—, C— or N- and C-terminal peptide/protein fusions in addition to (a) and/or (b).

The HA or HA domain fragment may also be made multifunctional by grafting the peptides derived from different screens of different loops against different targets into the same HA or HA domain fragment.

Peptides inserted into a loop of human serum albumin are CNTF or peptide fragments or peptide variants thereof. More particularly, the invention encompasses albumin fusion proteins which comprise peptide fragments or peptide variants at least 7, at least 8, at least 9, at least 10, at least 11, at least 12, at least 13, at least 14, at least 15, at least 20, at least 25, at least 30, at least 35, or at least 40 amino acids in length inserted into a loop of human serum albumin. The invention also encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids fused to the N-terminus of human serum albumin. The invention also encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids fused to the C-terminus of human serum albumin.

Generally, the albumin fusion proteins of the invention may have one HA-derived region and one CNTF protein-derived region. Multiple regions of each protein, however, may be used to make an albumin fusion protein of the invention. Similarly, more than one CNTF may be used to make an albumin fusion protein of the invention. For instance, a CNTF may be fused to both the N- and C-terminal ends of the HA. In such a configuration, the CNTF portions may be the same or different CNTF molecules. The structure of bifunctional albumin fusion proteins may be represented as: X-HA-Y or Y-HA-X or X—Y-HA or HA-X—Y or HA-Y—X-HA or HA-X—X-HA or HA Y—Y-HA or HA-X-HA-Y or X-HA-Y-HA or multiple combinations and/or inserting X and/or Y within the HA sequence at any location.

Bi- or multi-functional albumin fusion proteins may be prepared in various ratios depending on function, half-life etc.

Bi- or multi-functional albumin fusion proteins may also be prepared to target the CNTF portion of a fusion to a target organ or cell type via protein or peptide at the opposite terminus of HA.

As an alternative to the fusion of known therapeutic molecules, the peptides could be obtained by screening libraries constructed as fusions to the N—, C— or N- and C-termini of HA, or domain fragment of HA, of typically 6, 8, 12, or 25 or Xₙ (where X is an amino acid (aa) and n equals the number of residues) randomized amino acids, and in which all possible combinations of amino acids were represented. A particular advantage of this approach is that the peptides may be selected in situ on the HA molecule and the properties of the peptide would therefore be as selected for rather than, potentially, modified as might be the case for a peptide derived by any other method then being attached to HA.

Additionally, the albumin fusion proteins of the invention may include a linker peptide between the fused portions to provide greater physical separation between the moieties and thus maximize the accessibility of the CNTF portion, for instance, for binding to its cognate receptor. The linker peptide may consist of amino acids such that it is flexible or more rigid.

Therefore, as described above, the albumin fusion proteins of the invention may have the following formula R2-R1; R1-R2; R2-R1-R2; R2-L-R1-L-R2; R1-L-R2; R2-L-R1; or R1-L-R2-L-R1, wherein R1 is at least one Therapeutic protein, peptide or polypeptide sequence (including fragments or variants thereof), and not necessarily the same Therapeutic protein, L is a linker and R2 is a serum albumin sequence (including fragments or variants thereof). Exemplary linkers include (GGGGS)_{N} (SEQ ID NO:8) or (GGGS)_{N} (SEQ ID NO:9) or (GGS)_{N}, wherein N is an integer greater than or equal to 1 and wherein G represents glycine and S represents serine. When R1 is two or more Therapeutic proteins, peptides or polypeptide sequence, these sequences may optionally be connected by a linker.

In further embodiments, albumin fusion proteins of the invention comprising a CNTF protein have extended shelf-life or in vivo half-life or therapeutic activity compared to the shelf-life or in vivo half-life or therapeutic activity of the same CNTF when not fused to albumin. Shelf-life typically refers to the time period over which the therapeutic activity of a CNTF protein in solution or in some other storage formulation, is stable without undue loss of therapeutic activity. Many of the CNTF proteins are highly labile in their non-fused state. As described below, the typical shelf-life of these CNTF proteins is markedly prolonged upon incorporation into the albumin fusion protein of the invention.

Albumin fusion proteins of the invention with “prolonged” or “extended” shelf-life exhibit greater therapeutic activity relative to a standard that has been subjected to the same storage and handling conditions. The standard may be the non-fused full-length CNTF protein. When the CNTF portion of the albumin fusion protein is an analogue, a variant, or is otherwise altered or does not include the complete sequence for that protein, the prolongation of therapeutic activity may alternatively be compared to the non-fused equivalent of that analogue, variant, altered peptide or incomplete sequence. As an example, an albumin fusion protein of the invention may retain greater than about 100% of the therapeutic activity, or greater than about 105%, 110%, 120%, 130%, 150% or 200% of the therapeutic activity of a standard when subjected to the same storage and handling conditions as the standard when compared at a given time point. However, it is noted that the therapeutic activity depends on the CNTF protein's stability, and may be below 100%.

Shelf-life may also be assessed in terms of therapeutic activity remaining after storage, normalized to therapeutic activity when storage began. Albumin fusion proteins of the invention with prolonged or extended shelf-life as exhibited by prolonged or extended therapeutic activity may retain greater than about 50% of the therapeutic activity, about 60%, 70%, 80%, or 90% or more of the therapeutic activity of the equivalent non-fused CNTF when subjected to the same conditions.

### EXAMPLE 1

### Preparation of Albumin-fused AXOKINE®

CNTF was cloned from human genomic DNA by amplification of the two exons using primers

5′-CTCGGTACCCAGCTGACTTGTTTCCTGG-3′
and
5′-ATAGGATTCCGTAAGAGCAGTCAG-3′
for exon 1, and primer
5′-GTGAAGCATCAGGGCCTGAAC-3′
and
5′-CTCTCTAGAAGCAAGGAAGAGAGAAGGGAC-3′

for exon 2, respectively, using standard conditions. Both fragments were ligated under standard conditions, before being re-amplified by PCR using primers

5′-CTCGGTACCCAGCTGACTTGTTTCCTGG-3′
and
5′-CTCTCTAGAAGCAAGGAAGAGAGAAGGGAC-3′

and cloned into vector pCR4 (Invitrogen). To generate AXOKINE® as disclosed in Lambert et al. (PNAS 98:4652-4657; 2001) site-directed mutagenesis was employed to introduce C17A (TGT->GCT) and Q63R (CAG->AGA) mutations. DNA sequencing also revealed the presence of a silent T->C substitution V85V (GTT->GTC).

To create the C-terminal rHA-GS-AXOKINE® fusion the AXOKINE® cDNA was ligated to a cDNA encoding human albumin by mutagenic PCR using single stranded oligonucleotide primers

| | |
|---|---|
| MH32 | 5′-TGCCAAGCTTATTACCCAGTCTGATGAGAAGA- |
| | AATGAAACGAAGGTCATGG-3′ |
| and | |
| MH35 | 5′-TGGTGGATCCGGTGGTGCTTTCACAGAGCATTCACCG- |
| | CTGACCCC-3′ |

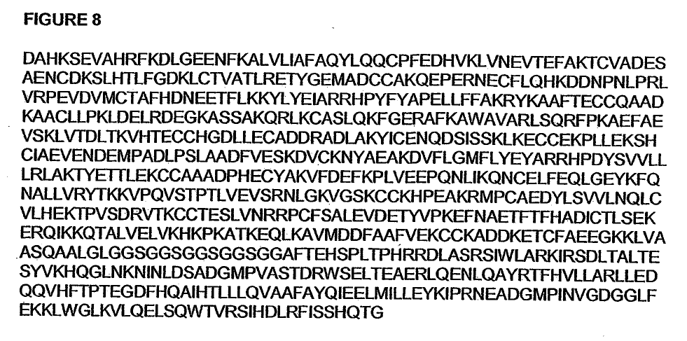

so as to introduce a 14 amino acid GS (-Gly-Gly-Ser-Gly-Gly-Ser-Gly-Gly-Ser-Gly-Gly-Ser-Gly-Gly-) peptide spacer. The amino acid sequence of the mature rHA-GS-AXOKINE® fusion is given in FIG. 8.

To create the C-terminal rHA-3×FLAG-AXOKINE® (cleavable AXOKINE®) fusion the AXOKINE® cDNA was ligated to a cDNA encoding human albumin by mutagenic PCR using single stranded oligonucleotide primers

MH32
5′-TGCCAAGCTTATTACCCAGTCTGATGAGAAGAAATG-
AAACGAAGGTCATGG-3′
and
CF83
5′-TCATGATATCGATTACAAGGATGACGATGACAAGG-
CTTTCACAGAGCATTCACCGCTGACCCCTCACCGTCGGGACCTCG-3′

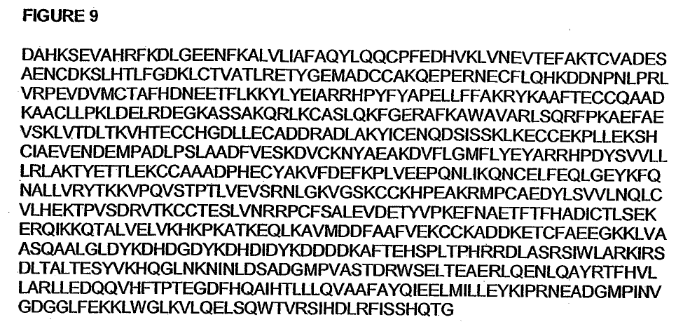

so as to introduce a 22 amino acid 3×FLAG (-Asp-Tyr-Lys-Asp-His-Asp-Gly-Asp-Tyr-Lys-Asp-His-Asp-Ile-Asp-Tyr-Lys-Asp-Asp-Asp-Asp-Lys-) peptide spacer (Sigma-Aldrich Company Ltd.) between the albumin and AXOKINE® sequences. The amino acid sequence of the mature C-terminal rHA-3×FLAG-AXOKINE® fusion is given in FIG. 9. The HSA/MFa-1 fusion secretion leader sequence disclosed in WO 90/01063 was provided to ensure secretion of the fusion protein.

To create the N-terminal AXOKINE®-GS-rHA fusion the AXOKINE® cDNA was ligated to a cDNA encoding human albumin by mutagenic PCR using single stranded oligonucleotide primers

| | |
|---|---|
| MH33 | 5′-ATGCAGATCTTTGGATAAGAGAGCTTTCACAGAG- |
| | CATTCACCGCTGACCCC-3′ |
| and | |
| MH36 | 5′-CACCGGATCCACCCCCAGTCTGATGAGAAG- |
| | AAATGAAACGAAGGTCATGG-3′ |

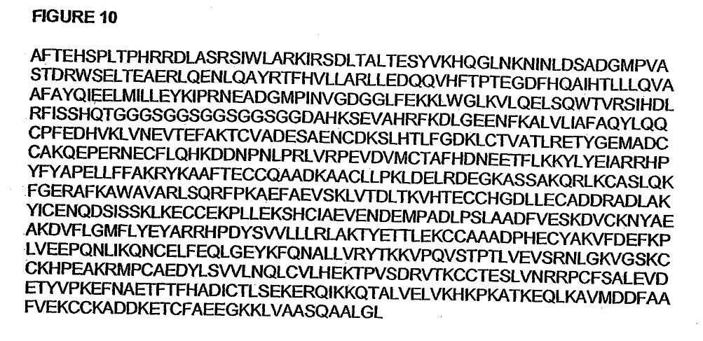

so as to introduce either a 14 amino acid GS (-Gly-Gly-Ser-Gly-Gly-Ser-Gly-Gly-Ser-Gly-Gly-Ser-Gly-Gly-) peptide spacer between the AXOKINE® and albumin sequences. The amino acid sequence of the mature AXOKINE-GS-rHA fusion is given in FIG. 10.

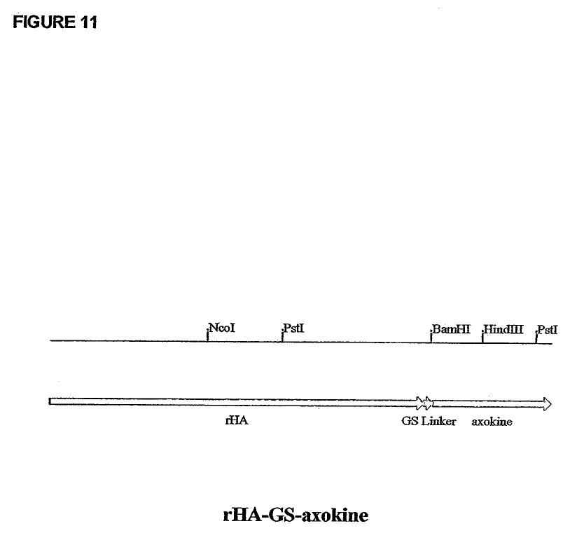

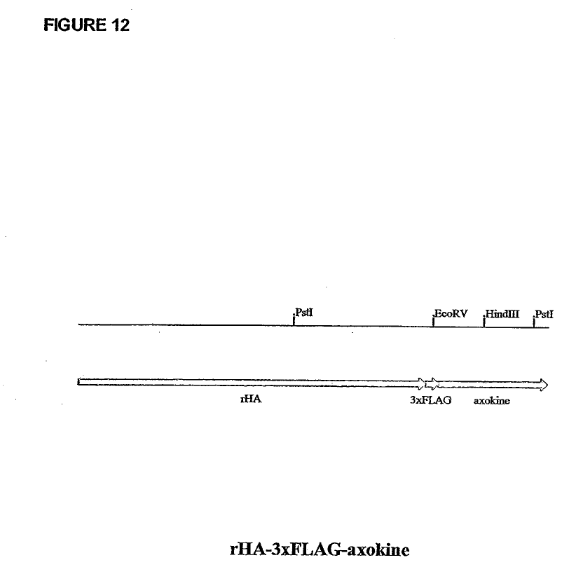

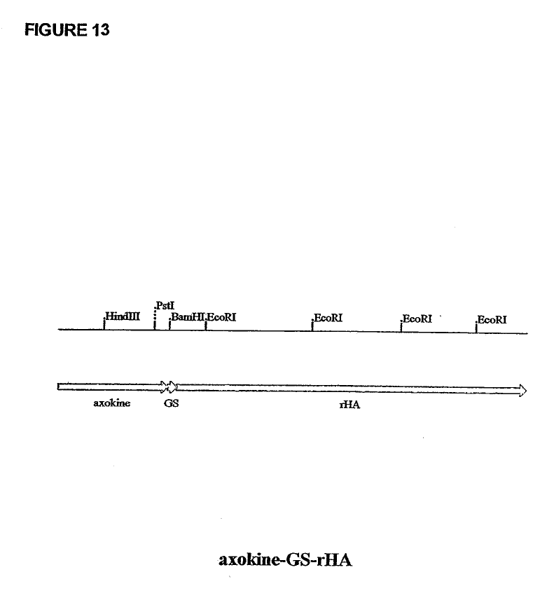

Maps of the rHA-GS-AXOKINE® sequence, the rHA-3×FLAG-AXOKINE® sequence and the AXOKINE®-GS-rHA sequence are shown in FIGS. 11, 12 and 13, respectively.

The yeast PRB1 promoter and the yeast ADH1 terminator provided appropriate transcription promoter and transcription terminator sequences, respectively as previously disclosed in WO 00/44772 and described by Sleep, D., et al. (1991) Bio/Technology 9, 183-187. Appropriate vector sequences were provide by a “disintegration” plasmid pSAC35 generally disclosed in EP-A-286 424 and described by Sleep, D., et al. (1991) Bio/Technology 9, 183-187.

The rHA fusions were expressed and shake flask culture expression level determined.

### EXAMPLE 2

### Purification

The C-Terminal AXOKINE® contained high levels of clipped material. It was purified using the standard rHA SP-FF conditions (See U.S. Pat. No. 6,034,221) but in a negative mode whereby the fusion was in the flow through. The flow through was adjusted to pH8 and 2.5 mS.cm⁻¹ and loaded on a standard rHA DE-FF equilibrated in 15 mM potassium tetraborate. As for the SP-FF the DEFF was operated in a negative mode. The conductivity of the DE-FF flow through was increased to 15 mS.cm⁻¹ and the material then purified using standard rHA DBA chromatography with an extra elution of 50 mM octanoate. The eluate was then concentrated and diafiltered against 5 mM phosphate pH8.3.

The N-Terminal AXOKINE® contained some clipped material. It was purified using the standard rHA SP-FF conditions but in a negative mode whereby the fusion was in the flow through. The flow through was adjusted to pH 8 and 2.5 mS.cm⁻¹ and loaded on a standard rHA DE-FF equilibrated in 15 mM potassium tetraborate. In this instance, a proportion of the fusion bound and was eluted in the standard elution containing 200 mM NaCl. The conductivity of the eluate was reduced to 15 mS.cm⁻¹ and the material purified using standard rHA DBA chromatography with an extra elution of 50 mM octanoate. The eluate was then concentrated and diafiltered against 5 mM phosphate pH8.3.

The cleavable AXOKINE® contained high levels of clipped material. It was purified using the standard rHA SP-FF conditions but in a negative mode whereby the fusion was in the flow through. The flow through was adjusted to pH 8 and 2.5 mS.cm⁻¹ and loaded on a standard rHA DE-FF equilibrated in 15 mM potassium tetraborate. As for the SP-FF this was operated in a negative mode. The conductivity of the flow through was increased to 15 mS.cm⁻¹ and the material purified using standard rHA DBA chromatography with an extra elution of 50 mM octanoate. The material was then concentrated and diafiltered into cleavage buffer. Cleavage was performed overnight at room temperature and the enterokinase removed using Ekapture gel. The material was then concentrated and diafiltered against 5 mM phosphate pH8.3.

### EXAMPLE 3

### Pharmacokinetics

Assessing the half-life and bioavailability of N-terminal and C-terminal albumin-fused AXOKINE® versus non-fused AXOKINE® and assessing additional pharmacokinetic parameters of N-terminal and C-terminal albumin-fused AXOKINE® versus non-fused AXOKINE®.

Administration Protocol:

| | |
|---|---|
| Test article 1: | Non-fused AXOKINE ® |
| Application volume: | 0.33 mL/kg |
| Single dose/route: | 10 μg/kg i.v. or s.c. |
| Frequency: | 1 x (t = 0) |
| Test article 2: | N-terminal albumin-fused AXOKINE ® |
| Application volume: | 0.33 mL/kg |
| Single dose/route: | 40 μg/kg i.v. or s.c. |
| Frequency: | 1 x (t = 0) |
| Test article 3: | C-terminal albumin-fused AXOKINE ® |
| Application volume: | 0.33 mL/kg |
| Single dose/route: | 40 μg/kg i.v. or s.c. |
| Frequency: | 1 x (t = 0) |

Study Design

TABLE 1
Treatment groups

| No. | Treatment | Dose/schedule/route | N (M/F) |
|---|---|---|---|
| 1 | Cleavable | 10 μg/kg/single injection/i.v. | 2 m + 2 f |
| | AXOKINE ® | | |
| 2 | C-term. albumin-fused | 40 μg/kg/single injection/i.v. | 2 m + 2 f |
| | AXOKINE ® | | |
| 3 | N-term. albumin-fused | 40 μg/kg/single injection/i.v. | 2 m + 2 f |
| | AXOKINE ® | | |
| 4 | Cleavable | 10 μg/kg/single injection/s.c. | 2 m + 2 f |
| | AXOKINE ® | | |
| 5 | C-term. albumin-fused | 40 μg/kg/single injection/s.c. | 2 m + 2 f |
| | AXOKINE ® | | |
| 6 | N-term. albumin-fused | 40 μg/kg/single injection/s.c. | 2 m + 2 f |
| | AXOKINE ® | | |

Experimental Animals

| | |
|---|---|
| Species/Strain: | rabbits |
| Sex/Age: | 12 males, 12 females; 3-4 months |
| No. total: | 24 |
| Supplier: | Fa. Bauer (Neuenstein-Lohe, Germany) |

Animal Model

Two male and two female rabbits per group received cleavable AXOKINE® (10 μg/kg), C-terminal albumin-fused AXOKINE® (40 μg/kg), or N-terminal albumin-fused AXOKINE® (40 μg/kg) by a single i.v. or s.c. injection on day 0. Blood samples were drawn for the determination of the respective antigen levels at baseline and at 5 min, 10 min, 20 min, 30 min, 45 min, 1 h, 2 h, 4 h, 8 h, 24 h (1 d), 48 h (2 d), 72 h (3 d), 5 d, 7 d, 9 d, 11 d, and 14 d after i.v. administration of the respective test substance and at baseline, 30 min, 1 h, 2 h, 4 h, 8 h, 24 h (1 d), 48 h (2 d), 72 h (3 d), 5 d, 7 d, 9 d, 11 d and 14 d following s.c. injection. Plasma levels of AXOKINE® and albumin-fused AXOKINE® were determined by ELISA.

Pharmacokinetic (PK) Variables:

Elimination half-life, area under the plasma concentration time curve up to Day 14 (AUC₀₋₁₄), maximum concentration (Cₘₐₓ). Area under the concentration time curve extrapolated to infinity (AUC_{0-∞}), time of maximum concentration (tₘₐₓ), mean residence time, half-lives of absorption and distribution (if applicable), clearance, volume of distribution.

Analytical Methods:

ELISA determination of non-fused AXOKINE® plasma concentration was performed using a monoclonal mouse anti-hu CNTF-antibody (R&D Systems, clone no. 21809.111) in combination with a biotinylated polyclonal goat anti-hu CNTF antibody (R&D Systems, cat. no. BAF257). Human CNTF was used as standard according to the ELISA kit description.

ELISA determination of albumin-fused AXOKINE® plasma concentration was performed using a monoclonal anti-hu albumin antibody (Aventis Behring GmbH, Laboratory) in combination with a biotinylated polyclonal goat anti-hu. CNTF antibody (R&D Systems, cat. no. BAF257). The respective albumin-fused AXOKINE® served for generation of the standard curve.

Using the commercially available human CNTF ELISA (R&D Systems), it was not possible to detect the albumin-fused AXOKINE®, probably due to sterical interference of the albumin with the binding of the anti-CNTF antibodies.

As a solution, an internal anti-albumin assay was established, using an anti-albumin monoclonal antibody as capture antibody, where this antibody was coupled to the plate. As the next step, the commercially available CNTF antibody from R&D Systems was used as detection antibody for albumin-fused AXOKINE®.

Analysis of Individual Plasma Levels

The plasma concentration-time profiles of C- and N-terminal albumin-fused AXOKINE® and non-fused AXOKINE® were analyzed per animal by means of nonlinear regression. An exponential model was fitted to the data by the method of least squares. For the profiles following i.v. administration, an open two-compartment model was used. For the profiles following s.c. administration, an open one-compartment model with first-order input and lag time was used. For the i.v. model, a weighting factor of 1/(predicted concentration)² was applied.

The AUC was calculated a) using the linear trapezoidal rule up to the last measured value (AUC₀₋₁₄) and b) completing AUC₀₋₁₄ by extrapolation for the period between Day 14 and infinity (AUC_{0-∞}).

### SUMMARY AND COMPARATIVE ANALYSES

Individual PK results were summarized descriptively per treatment and route of application (minimum, median, maximum, mean, standard deviation).

A two-way analysis of variance was carried out for elimination half-life, AUC and Cₘₐₓ (all log-transformed). Fixed factors were sex and treatment group. Appropriate contrasts between treatment groups were evaluated. The possibility of unequal variances was also taken into account.

For the purpose of this analysis it was assumed that In (half-life), In (AUC) and In (Cₘₐₓ) each follow a normal distribution.

Elimination half-lives were compared between substances, bioavailabilities in terms of AUC and Cₘₐₓ were compared between the routes of administration for the albumin-fused AXOKINE® groups at an alpha level of 0.1 using two-sided 90% confidence intervals.

Results

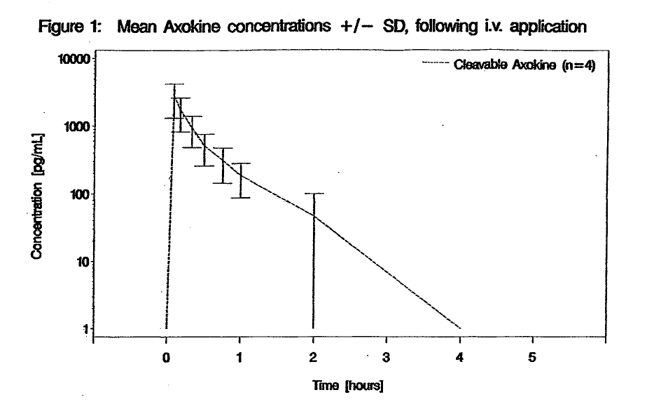

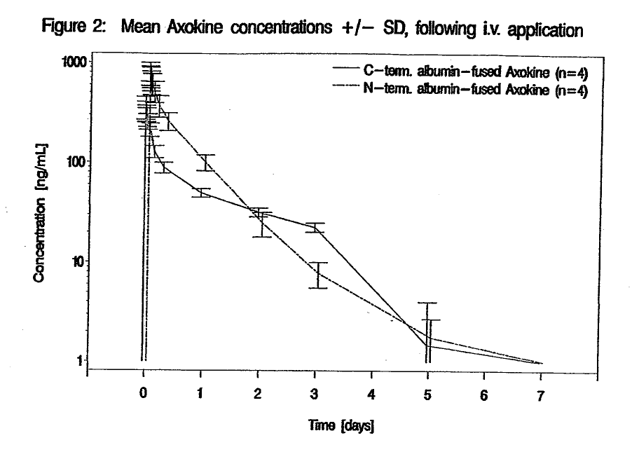

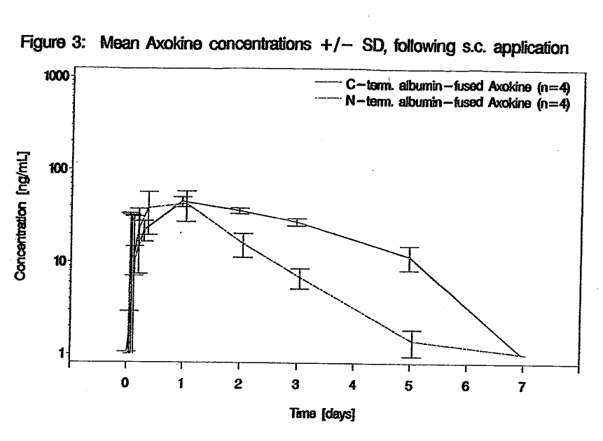

The means and standard deviations of the AXOKINE® concentrations at every time point are shown in FIG. 1 for the i.v. treated non-fused AXOKINE® group, in FIG. 2 for the i.v. treated albumin-fused AXOKINE® groups, and in FIG. 3 for the s.c. treated albumin-fused AXOKINE® groups. For the s.c. treated non-fused AXOKINE® group, no concentrations could be measured.

In the animals treated intravenously with non-fused AXOKINE®, the levels fell below 1 pg/mL 4 hours after injection. In the albumin-fused AXOKINE® groups, the levels stayed above 1 ng/mL for 7 days. In the animals treated subcutaneously with the albumin-fused AXOKINE® products, the levels reached their peaks after about 1 day and stayed above 1 ng/mL for 7 days. The pharmacokinetic results are presented in Table 2 for the i.v. treated groups and in Table 3 for the s.c. treated groups. The results for non-fused AXOKINE® were converted to the same units as the albumin-fused AXOKINE® groups, but, with the exception of half-lives and mean residence times, cannot be compared with these because of the different assay methods.

TABLE 2
Pharmacokinetic results following i.v. administration

| | | | C-terminal | N-terminal |
|---|---|---|---|---|
| | | Non-fused | albumin-fused | albumin-fused |
| | | AXOKINE ® | AXOKINE ® | AXOKINE ® |
| | N | 4 | 4 | 4 |
| Initial half- | Mean | 0.11 | 2.32 | 7.08 |
| life (hr) | Std Dev | 0.02 | 1.14 | 1.84 |
| | Median | 0.11 | 2.38 | 6.55 |
| | Range | 0.09-0.13 | 0.88-3.66 | 5.50-9.74 |
| Terminal | Mean | 0.49 | 36.2 | 23.5 |
| half-life (hr) | Std Dev | 0.19 | 15.4 | 10.7 |
| | Median | 0.50 | 36.7 | 19.9 |
| | Range | 0.30-0.66 | 17.5-54.0 | 15.1-39.1 |
| Mean | Mean | 0.44 | 43.7 | 17.0 |
| residence | Std Dev | 0.10 | 17.6 | 2.7 |
| time (hr) | Median | 0.45 | 42.9 | 15.9 |
| | Range | 0.34-0.55 | 23.2-65.7 | 15.4-21.0 |
| AUC₀₋₁₄ = | Mean | 0.97 | 4,628 | 8,389 |
| AUC_{0-∞} | Std Dev | 0.54 | 413 | 1,693 |
| (hr · ng/mL) | Median | 0.93 | 4,600 | 8,394 |
| | Range | 0.44-1.59 | 4,272-5,039 | 6,475-10,293 |
| | Geom. | 0.85 | 4,614 | 8,259 |
| | mean | | | |
| | SF* | 1.84 | 1.09 | 1.23 |
| Cₘₐₓ | Mean | 2.77 | 362 | 820 |
| (ng/mL) | Std Dev | 1.45 | 94 | 178 |
| | Median | 2.68 | 356 | 803 |
| | Range | 1.35-4.35 | 275-463 | 632-1,041 |
| Total | Mean | 11,774 | 2.06 | 1.18 |
| clearance | Std Dev | 6,709 | 0.33 | 0.24 |
| (mL/hr/kg) | Median | 10,735 | 2.10 | 1.13 |
| | Range | 5,629-19,995 | −1.68-2.36 | 0.95-1.51 |
| Total | Mean | 4,745 | 87.4 | 20.0 |
| volume of | Std Dev | 1,728 | 30.0 | 4.5 |
| distribution | Median | 4,563 | 85.7 | 20.3 |
| (mL/kg) | Range | 3,089-6,762 | 53.3-124.8 | 15.1-24.5 |

*SF = scatter factor = exp[standard deviation (log-transformed values)]

TABLE 3
Pharmacokinetic results following s.c. administration

| | | C-terminal | |
|---|---|---|---|
| | | albumin-fused | N-terminal albumin- |
| | | AXOKINE ® | fused AXOKINE ® |
| | N | 4 | 4 |
| Lag time (hr) | Mean | 1.75 | 3.47 |
| | Std Dev | 0.75 | 3.78 |
| | Median | 1.54 | 3.42 |
| | Range | 1.11-2.80 | 0.00-7.05 |
| Absorption half- | Mean | 13.3 | 6.01 |
| life (hr) | Std Dev | 5.5 | 5.52 |
| | Median | 10.9 | 5.03 |
| | Range | 9.9-21.4 | 1.35-12.64 |
| Terminal half-life | Mean | 30.5 | 15.4 |
| (hr) | Std Dev | 6.8 | 1.7 |
| | Median | 31.8 | 15.9 |
| | Range | 21.3-37.4 | 13.0-16.9 |
| AUC₀₋₁₄ = AUC_{0-∞} | Mean | 3,534 | 1,986 |
| (hr · ng/mL) | Std Dev | 383 | 610 |
| | Median | 3,598 | 1,916 |
| | Range | 3,011-3,931 | 1,323-2,788 |
| | Geom. mean | 3,518 | 1,917 |
| | SF* | 1.12 | 1.36 |
| Cₘₐₓ (ng/mL) | Mean | 44.6 | 45.5 |
| | Std Dev | 5.5 | 14.2 |
| | Median | 45.6 | 45.2 |
| | Range | 37.3-50.1 | 28.5-63.0 |
| tₘₐₓ (hr) | Mean | 24 | 20 |
| | Std Dev | 0 | 8 |
| | Median | 24 | 24 |
| | Range | 24-24 | 8-24 |
| Relative total | Mean | 2.75 | 5.23 |
| clearance | Std Dev | 0.33 | 1.66 |
| (mL/hr/kg) | Median | 2.68 | 4.93 |
| | Range | 2.42-3.22 | 3.54-7.50 |
| Relative total | Mean | 118.8 | 113.6 |
| volume of | Std Dev | 14.0 | 25.0 |
| distribution | Median | 122.9 | 115.2 |
| (mL/kg) | Range | 98.6-130.7 | 83.4-140.5 |

*SF = scatter factor = exp[standard deviation (log-transformed values)]

Table 4 shows the results of the analyses of variance regarding the elimination half-life. The differences between non-fused and albumin-fused AXOKINE® following i.v. injection were highly significant. The sex of the animals did not have a significant influence on the half-life.

TABLE 4
Comparison of elimination half-lives between substances

| | | Elimination |
|---|---|---|
| Route | Parameter | half-life |
| i.v. | Estimated ratio (C-terminal albumin-fused | 72.4 |
| | AXOKINE ®/cleavable AXOKINE ®) | |
| | 90% confidence limits | 38.5-136.4 |
| i.v. | Estimated ratio (N-terminal albumin-fused | 47.5 |
| | AXOKINE ®/cleavable AXOKINE ®) | |
| | 90% confidence limits | 24.4-92.8 |

Table 5 shows the results of the analyses of variance regarding the absolute bioavailability. For both albumin-fused products, the differences between the two routes of application were not statistically significant with respect to elimination half-life. The differences regarding AUC and Cₘₐₓ were highly significant.

TABLE 5
Comparison of bioavailabilities between routes of application

| | | Elimination | | |
|---|---|---|---|---|
| Substance | Parameter | half-life | AUC₀₋₁₄ | Cₘₐₓ |
| C-terminal | Estimated ratio | 0.89 | 0.76 | 0.13 |
| albumin-fused | (s.c./i.v.) | | | |
| AXOKINE ® | 90% confidence | 0.52-1.55 | 0.69-0.84 | 0.10-0.16 |
| | limits | | | |
| N-terminal | Estimated ratio | 0.70 | 0.23 | 0.05 |
| albumin-fused | (s.c./i.v.) | | | |
| AXOKINE ® | 90% confidence | 0.42-1.17 | 0.15-0.37 | 0.03-0.11 |
| | limits | | | |

The values for area under the curve and maximum plasma levels of non-fused AXOKINE® cannot be compared directly to those of N- and C-terminal albumin-fused AXOKINE®. In contrast to this, the comparison of the half-lives is valid.

Both albumin-fused AXOKINE® preparations showed a markedly prolonged elimination from plasma after i.v. application compared to non-fused AXOKINE®. C-terminal albumin-fused AXOKINE® showed an average elimination half-life that was 72 times longer than that of non-fused AXOKINE®. N-terminal albumin-fused AXOKINE® showed an average elimination half-life that was 48 times longer than that of non-fused AXOKINE®.

In terms of AUC, the absolute bioavailability after s.c. injection was 76% for C-terminal albumin-fused AXOKINE® and 23% for N-terminal albumin-fused AXOKINE®. Since plasma levels of non-fused AXOKINE® were below the detection limit after s.c. application, the comparison with the i.v. application could not be made.

### EXAMPLE 4

### Pharmacodynamics

The purpose of this example was to assess the efficacy of N- and C-terminal albumin-fused AXOKINE® as compared to placebo or non-fused AXOKINE® in reduction of body weight in leptin-deficient or dietary-induced obese mice.

Study Design of Pharmacodynamic Animal Study, Part I

This study was designed as a randomised, partly blinded, parallel, 13-armed trial with two experimental settings (leptin-deficiency induced obesity versus dietary-induced obesity) including a total of 70 female C57BL/6Jlep^{ob} (ob/ob), and 41 male and 41 female C57BL/6J mice.

Experimental Animals

C57BL/6Jlep^{ob} (ob/ob) mice were fed standard diet for approximately 3 months. During this time, C57BL/6Jlep^{ob} (ob/ob) mice strongly increased weight due to uncontrolled food intake associated with leptin-deficiency. In wild-type C57BL/6J mice, obesity was induced by feeding with high caloric food containing 45% of fat. Body weight was recorded weekly during this phase of obesity induction preceding therapeutic treatment. After a mean weight increase to at least 130% of baseline, treatment with the test substances was started. Test substances (Non-fused AXOKINE®, albumin-fused AXOKINE®, placebo) were administered by daily subcutaneous injections over a period of seven days. During the treatment phase, body weights were determined daily. The mean weight loss as compared to baseline and placebo was calculated to assess the relative efficacy of the test substances.

Study Medication and Dosage

| | |
|---|---|
| Test article 1: | Placebo (5 mM phosphate buffer at pH 8.3) |
| Endotoxin content: | 0.007 EU/mL |
| Stock concentration: | n.a. |
| Application volume: | 250 μl^{a} |
| Single dose/route: | n.a./s.c. |
| Frequency: | seven daily injections |
| Test article 2: | Non-fused AXOKINE ® |
| Endotoxin content: | 14.9 EU/m2L |
| Stock concentration: | 0.1 mg/mL |
| Application volume: | 250 μl^{a} |
| Single dose/route: | according to table 1 & 2/s.c. |
| Frequency: | seven daily injections |
| Test article 3: | N-terminal albumin-fused AXOKINE ® |
| Endotoxin content: | 1.8 EU/mL |
| Stock concentration: | 5 mg/mL |
| Application volume: | 250 μl^{a} |
| Single dose/route: | according to table 1 & 2/s.c. |
| Frequency: | seven daily injections |
| Test article 4: | C-terminal albumin-fused AXOKINE ® |
| Endotoxin content: | 64 EU/mL & 32 EU/mL |
| Stock concentration: | 0.2 mg/mL |
| Application volume: | 250 μl^{a} |
| Single dose/route: | according to table 1 & 2/s.c. |
| Frequency: | seven daily injections |

^{a}All mice received 250 μl test substance at treatment day 1 (Day 83), then, dosing was adjusted to body weight changes by adjustment of the administered volume. Mice in group 13 (1200 μg/kg C-terminal AXOKINE ®) received approximately 390 μl at Day 83.

TABLE 6
Treatment groups C57BL/6Jlep^{ob} (ob/ob) mice

| No. | Treatment | Dose/volume/schedule/route | N (m/f) |
|---|---|---|---|
| 1 | Placebo | —/250 μl/7 daily injections/s.c. | 10 f |
| 2 | Non-fused | 10 μg/kg/250 μl/7 daily injections/s.c. | 5 f |
| | AXOKINE ® | | |
| 3 | Non-fused | 100 μg/kg/250 μl/7 daily injections/ | 5 f |
| | AXOKINE ® | s.c. | |
| 4 | Non-fused | 300 μg/kg on Days 1-2, 200 μ/kg on | 5 f |
| | AXOKINE ® | treatment days 3-7/250 μl/7 daily | |
| | | injections/s.c. | |
| 5 | N-albumin-fused | 40 μg/kg/250 μl/7 daily injections/s.c. | 5 f |
| | AXOKINE ® | | |
| 6 | N-albumin-fused | 280 μg/kg on Days 1-2, 200 μl/kg on | 5 f |
| | AXOKINE ® | treatment days 3-7/250 μl/7 daily | |
| | | injections/s.c. | |
| 7 | N-albumin-fused | 400 μg/kg/250 μl/7 daily injections/ | 5 f |
| | AXOKINE ® | s.c. | |
| 8 | N-albumin-fused | 1200 μg/kg on Days 1-2, 800 μ/kg on | 5 f |
| | AXOKINE ® | treatment days 3-7/250 μl/7 daily | |
| | | injections/s.c. | |
| 9 | C-albumin-fused | 40 μg/kg/250 μl/7 daily injections/s.c. | 5 f |
| | AXOKINE ® | | |
| 10 | C-albumin-fused | 280 μg/kg on Days 1-2, 200 μ/kg on | 5 f |
| | AXOKINE ® | treatment days 3-7/250 μl/7 daily | |
| | | injections/s.c. | |
| 11 | C-albumin-fused | 400 μg/kg/250 μl/7 daily injections/ | 5 f |
| | AXOKINE ® | s.c. | |
| 12 | C-albumin-fused | 1200 μg/kg on Days 1-2, 800 μ/kg on | 5 f |
| | AXOKINE ® | treatment days 3-7/390-250 μl/7 daily | |
| | | injections/s.c. | |

TABLE 7
Treatment groups C57BL/6J mice

| No. | Treatment | Dose/volume/schedule/route | N (m/f) |
|---|---|---|---|
| 1 | Placebo | —/250 μl/7 daily injections/s.c. | 5m/5f |
| 2 | Non-fused | 10 μg/kg/250 μl/7 daily | 3 m/3 f |
| | AXOKINE ® | injections/s.c. | |
| 3 | Non-fused | 100 μg/kg/250 μl/7 daily | 3 m/3 f |
| | AXOKINE ® | injections/s.c. | |
| 4 | Non-fused | 300 μg/kg on Days 1-2, 200 μg/kg on | 3 m/3 f |
| | AXOKINE ® | treatment days 3-7/250 μl/7 daily | |
| | | injections/s.c. | |
| 5 | N-albumin-fused | 40 μg/kg/250 μl/7 daily injections/s.c. | 3 m/3 f |
| | AXOKINE ® | | |
| 6 | N-albumin-fused | 280 μg/kg on Days 1-2, 200 μg/kg on | 3 m/3 f |
| | AXOKINE ® | treatment days 3-7/250 μl/7 daily | |
| | | injections/s.c. | |
| 7 | N-albumin-fused | 400 μg/kg/250 μl/7 daily | 3 m/3 f |
| | AXOKINE ® | injections/s.c. | |
| 8 | N-albumin-fused | 1200 μg/kg on Days 1-2, 800 μg/kg | 3 m/3 f |
| | AXOKINE ® | on treatment days 3-7/250 μl/7 daily | |
| | | injections/s.c. | |
| 9 | C-albumin-fused | 40 μg/kg/250 μl/7 daily injections/s.c. | 3 m/3 f |
| | AXOKINE ® | | |
| 10 | C-albumin-fused | 280 μg/kg on Days 1-2, 200 μg/kg on | 3 m/3 f |
| | AXOKINE ® | treatment days 3-7/250 μl/7 daily | |
| | | injections/s.c. | |
| 11 | C-albumin-fused | 400 μg/kg/250 μl/7 daily | 3 m/3 f |
| | AXOKINE ® | injections/s.c. | |
| 12 | C-albumin-fused | 1200 μg/kg on Days 1-2, 800 μg/kg | 3 m/3 f |
| | AXOKINE ® | on treatment days 3-7/390-250 μl/7 | |
| | | daily injections/s.c. | |

The following dose reductions had to be made for both, ob/ob as well as wildtype mice:

Non-fused AXOKINE® from Delta: from 300 μg/kg on Day 1-2 to 200 μg/kg on Day 3-7

N, C-terminal AXOKINE®: from 280 μg/kg on Day 1-2 to 200 μg/kg on Day 3-7

N, C-terminal AXOKINE®: from 1200 μg/kg on Day 1-2 to 800 μg/kg on Day 3-7

Randomization was done according to the randomization list, separately for C57BL/6Jlep^{ob} (ob/ob) and for C57BL/6J mice. After the mice were randomised to cages, cages were randomised to treatment.

Efficacy variables: Bodyweight (determined daily from Day 0-7).

Analytical Methods

Body weights were recorded by weighing of conscious animals.

Statistical Methods

Primary efficacy variable: Body weight difference between Day 7 and Day 0 and up to Day 102. Separated for C57BL/6Jlep^{ob} (ob/ob) and for C57BL/6J mice dose-response relationships for non-fused AXOKINE®, N-terminal albumin-fused AXOKINE®, and C-terminal albumin-fused AXOKINE® were analyzed within one analysis of variance model:

Successive comparison of the different doses with placebo using contrasts (e.g. Helmert or reverse Helmert contrasts) in order to identify the minimal effective dose. Comparison of pairs of equimolar doses using 2-sided t-tests and 2-sided 95% confidence intervals for the difference.

An overall assessment of N-terminal albumin-fused AXOKINE® and C-terminal albumin-fused AXOKINE® with regard to nonfused AXOKINE® was done by means of a parallel line assay with log-transformed doses. The derived potency was supplemented by a 95% confidence interval.

Results

Statistical Analysis of primary endpoint

Endpoint: Body weight change (g) from Day 82 to Day 91, 92, 93, 94, 95, 96,102

Statistics: F-tests within ANOVA in ordered hypotheses families. Starting on Day 92 a hypothesis is rejected provided the corresponding F-test is significant and the preceding hypothesis has also been rejected.

Reference: Bauer P: Multiple tests in clinical trials. Statistics in Medicine, 10:871-890, 1991

Weight Reduction in ob/ob mice

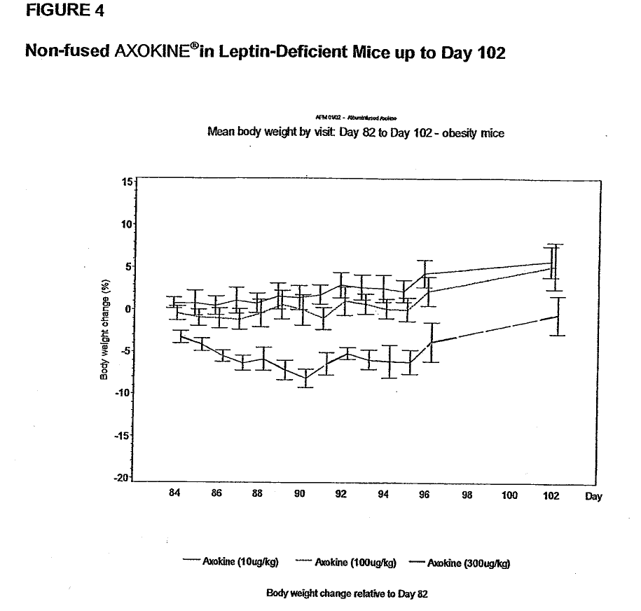

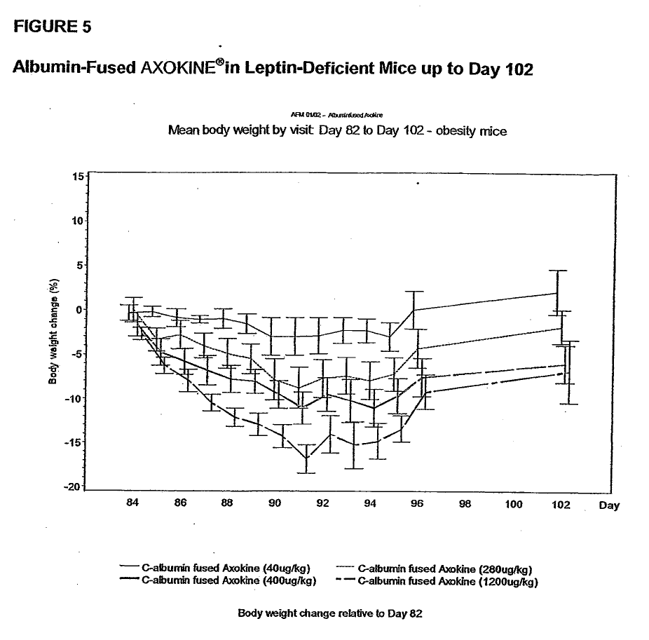

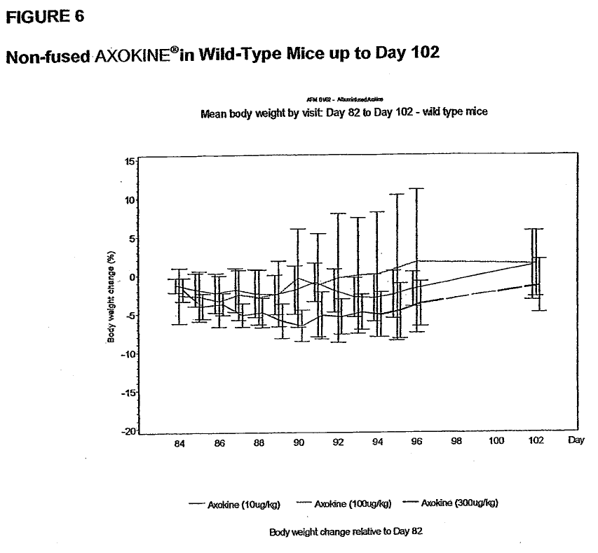

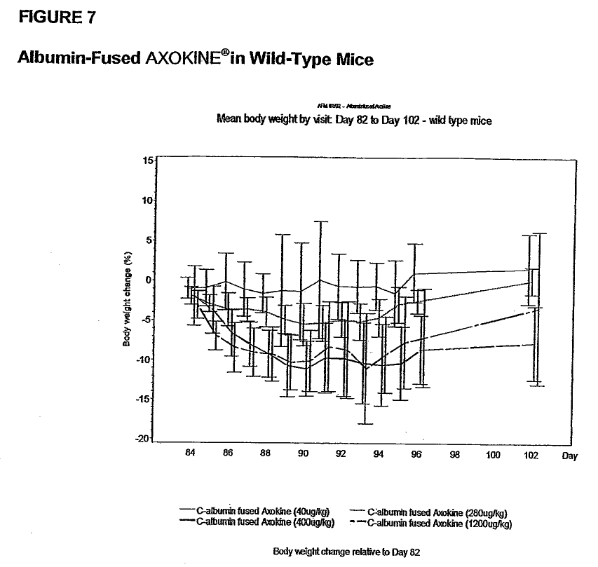

FIGS. 4, 5, 6 and 7 compare equimolar doses of the non-fused AXOKINE® with albumin fused AXOKINE® in leptin deficient mice.

In summary, the pharmacodynamic data show that in the leptin deficient mice, albumin fused AXOKINE® is statistically significant better than the non fused AXOKINE® for dose groups 11, 12, and 13. In wild type mice, the albumin fused AXOKINE® is statistically better compared to the non-fused AXOKINE® in group 12.

Study design of pharmacodynamic animal study, part II

The study was originally designed as a randomized, partly blinded, parallel, 11-armed trial with two experimental settings (leptin-deficiency induced obesity versus dietary-induced obesity) including a total of 82 female B6.V-Lep^{ob} (ob/ob) mice, and 41 male and 41 female C57BL/6J mice. Due to restricted availability of non-fused AXOKINE®, only selected treatment groups of leptin-deficient mice were included in the treatment phase of the study (Table 8).

TABLE 8
Treatment groups B6.V-Lep^{ob} (ob/ob) mice

| No. | Treatment | dose/volume/schedule/route | n (m/f) |
|---|---|---|---|
| 1 | Placebo | —/5 μl/g/7 daily injections/s.c. | 10 f |
| 2 | Non-fused | 100 μg/kg/5 μl/g/Days 1, 4, 7/s.c. | 6 f |
| | AXOKINE ® | | |
| 3 | Non-fused | 300 μg/kg/5 μl/g/Days 1, 4, 7/s.c. | 6 f |
| | AXOKINE ® | | |
| 4 | Non-fused | 100 μg/kg/5 μl/g/7 daily injections/ | 6 f |
| | AXOKINE ® | s.c. | |
| 5 | Non-fused | 300 μg/kg/5 μl/g/7 daily injections/ | 6 f |
| | AXOKINE ® | s.c. | |
| 6 | C-albumin-fused | 400 μg/kg/5 μl/g/Days 1, 4, 7/s.c. | 6 f |
| | AXOKINE ® | | |
| 7 | C-albumin-fused | 1200 μg/kg/5 μl/g/Days 1, 4, 7/s.c. | 6 f |
| | AXOKINE ® | | |
| 8 | C-albumin-fused | 3600 μg/kg/10 μl/g/Days 1, 4, 7/ | 6 f |
| | AXOKINE ® | s.c. | |
| 9 | C-albumin-fused | 400 μg/kg/5 μl/g/7 daily injections/ | 6 f |
| | AXOKINE ® | s.c. | |
| 10 | C-albumin-fused | 1200 μg/kg/5 μl/g/7 daily injections/ | 6 f |
| | AXOKINE ® | s.c. | |
| 11 | C-albumin-fused | 1200 μg/kg/5 μl/g/7 daily injections/ | 6 f |
| | AXOKINE ® | s.c. | |
| | (stability: 14 days | | |
| | at room | | |
| | temperature) | | |

Schedule

B6.V-Lep^{ob} mice were fed standard diet until Day 80 and increased weight. Treatment with either non-fused AXOKINE® or C-albumin-fused AXOKINE® started on Day 81 either for seven consecutive days (Days 81, 82, 83, 84, 85, 86, 87) or only on Days 1, 4, 7 (Days 81, 84, 87).

Body weight was assessed until 21 days post-treatment cessation (Day 108). Body weight changes and pertaining analyses were related to the weight on Day 81.

The corresponding timepoints are summarized in the table below:

TABLE 9
Treatment schedule B6.V-Lep^{ob} (ob/ob) mice

| Study Day | Treatment day |
|---|---|
| Day 81 | Day 1 |
| Day 84 | Day 4 |
| Day 87 | Day 7 |
| Day 101 | Day 21 |
| Day 108 | Day 28 |

### Administration of Test Articles

| | |
|---|---|
| Test article 1: | Placebo (5 mM phosphate buffer at pH 8.3) |
| Manufacturer: | Aventis Behring (Laboratory Dr. H. Metzner) |
| Batch No.: | — |
| Endotoxin content: | n.t. |
| Stock concentration: | n.a. |
| Application volume: | 5 μl/g |
| Single dose/route: | n.a./s.c. |
| Frequency: | seven daily injections |
| Test article 2: | Non-fused AXOKINE ® (Enterokinase-cleaved C- |
| | terminal albumin-fused AXOKINE ®) |
| Manufacturer: | Delta Biotechnology Ltd., Laboratory Dr. D. Sleep |
| Batch No.: | 1675#40 |
| Endotoxin content: | 18 EU/mL |
| Stock concentration: | approximately 0.1 mg/mL (assumption based on |
| | SDS PAGE with Coomassie staining compared to |
| | CNTF as a standard, Appendix B) |
| Application volume: | 5 μl/g |
| Single dose/route: | according to table 1/s.c. |
| Frequency: | single injections on days 1, 4, 7 or seven daily |
| | injections |
| Test article 3: | C-terminal albumin-fused AXOKINE ® |
| Manufacturer: | Delta Biotechnology Ltd., Laboratory Dr. D. Sleep, |
| | Aventis Behring GmbH, Laboratory Dr. H. Metzner |
| Batch No.: | 091002 |
| Endotoxin content: | 16 EU/mL |
| Stock concentration: | approximately 0.4 mg/mL (assumption based on |
| | SDS PAGE with Coomassie staining compared to |
| | HSA as a standard, Appendix B) |
| Application volume: | 5 μl/g^{a} |
| Single dose/route: | according to table 1/s.c. |
| Frequency: | single injections on days 1, 4, 7 or seven daily |
| | injections |
| Test article 4: | C-terminal albumin-fused AXOKINE ® stored at |
| | room temp. for 14 days |
| Manufacturer: | Delta Biotechnology Ltd., Laboratory Dr. D. Sleep, |
| | Aventis Behring GmbH, Laboratory Dr. H. Metzner |
| Batch No.: | 091002 |
| Endotoxin content: | 16 EU/mL |
| Stock concentration: | approximately 0.4 mg/mL (assumption based on |
| | SDS PAGE with Coomassie staining compared to |
| | HSA as a standard, Appendix B) |
| Application volume: | 5 μl/g^{a} |
| Single dose/route: | according to table 1/s.c. |
| Frequency: | single injections on days 1, 4, 7 or seven daily |
| | injections |

^{a}All mice received 5 μl/g test substance except mice treated with C-terminal AXOKINE ® 3600 μg/kg which received 10 μl/g.

### Animal Model

B6.V-Lep^{ob} (ob/ob) mice were fed standard diet for 12 weeks. During this time, mice strongly increased weight due to uncontrolled food intake associated with leptin-deficiency. Body weight was recorded weekly during this phase of obesity induction preceding therapeutic treatment with the exception of days 49-66, when animals were not weighed. Test substances (AXOKINE®, C-terminal albumin-fused AXOKINE®, placebo) were administered either by daily subcutaneous injections over a period of seven days or by three single injections at treatment Days 1, 4, 7. During the treatment phase, body weights were determined daily. Thereafter, body weight was recorded every other working day (i.e. 3 times per week) for 14 days and once more at 21 days post treatment (Day 28 after treatment start=study Day 108). The mean weight loss as compared to baseline and placebo was calculated to assess the relative efficacy of the test substances.

### Randomization

Randomization was done according to the randomization list. After randomization of mice to cages, cages were randomized to treatment.

### Efficacy Variables

| major: | Body weight change (g) from treatment Day 1 (study Day 81) to |
|---|---|
| | treatment Day 7 (Study Days 88, 87, 86, 85, 84, 83, and 82). |
| minor: | Body weight at Day 28 after start of treatment (Study Day 108). |
| | Body weight change (g) from Study Day 81 to Days 89, 91, 94, |
| | 96, 98, 101, 108. |

### Analytical Methods

Body weights were recorded by weighing of conscious animals.

Statistical Methods

F-tests within ANOVA in ordered hypotheses families. Starting on Day 88 and then proceeding downward, a hypothesis was rejected provided the corresponding F-test was significant (p≦0.05) and the preceding hypotheses had also been rejected (p≦0.05). The same procedure was applied starting on Day 89 upward until Day 108.

The procedure controlled the multiple level 0.05 within a set of comparisons, which consisted of the seven hypotheses related to the days.

Four blocks of analyses were conducted: Tables 10 and 11 compile test decisions for tests against placebo, i.e. active treatment groups (groups 2-11) were compared with placebo (group 1) in order to check model validity. While analyses of equimolar doses are provided in Tables 12 and 13, treatment schedules are compared in Tables 14 and 15. Finally, potency estimations are summarized in Table 16, using a parallel line assay on log-doses with Day 88 body weight change serving as response criterion. Tests on the suitability (i.e. linearity, parallelism) of the assay approach were not done.

Results

Effects on body weight

Study treatment was administered from Day 81 to Day 87.

Comparisons with Placebo

All groups receiving test substances showed a significant difference to placebo between Day 82 and Day 101 (Table 10 and 11).

TABLE 10
Test decisions for comparison against placebo
(i.e. validity of model —Day 88-82)

| | Day | | | | | | |
|---|---|---|---|---|---|---|---|
| Comparison | 88 | 87 | 86 | 85 | 84 | 83 | 82 |
| 2 vs. 1 | * | * | * | * | * | * | * |
| 3 vs. 1 | * | * | * | * | * | * | * |
| 4 vs. 1 | * | * | * | * | * | * | * |
| 5 vs. 1 | * | * | * | * | * | * | * |
| 6 vs. 1 | * | * | * | * | * | * | * |
| 7 vs. 1 | * | * | * | * | * | * | * |
| 8 vs. 1 | * | * | * | * | * | * | * |
| 9 vs. 1 | * | * | * | * | * | * | * |
| 10 vs. 1 | * | * | * | * | * | * | * |
| 11 vs. 1 | * | * | * | * | * | * | * |

note:
* (#) indicates that first (second) group shows a significantly (p < 0.05) larger weight reduction than second (first) group of the comparison.
“—” indicates absence of significance.

TABLE 11
Test decisions for comparison against placebo
(i.e. validity of model) - Days 89-108

| | Day | | | | | | |
|---|---|---|---|---|---|---|---|
| Comparison | 89 | 91 | 94 | 96 | 98 | 101 | 108 |
| 2 vs. 1 | * | * | * | * | * | * | — |
| 3 vs. 1 | * | * | * | * | * | * | — |
| 4 vs. 1 | * | * | * | * | * | * | * |
| 5 vs. 1 | * | * | * | * | * | * | * |
| 6 vs. 1 | * | * | * | * | * | * | — |
| 7 vs. 1 | * | * | * | * | * | * | * |
| 8 vs. 1 | * | * | * | * | * | * | * |
| 9 vs. 1 | * | * | * | * | * | * | * |
| 10 vs. 1 | * | * | * | * | * | * | * |
| 11 vs. 1 | * | * | * | * | * | * | * |

note:
* (#) indicates that first (second) group shows a significantly (p < 0.05) larger weight reduction than second (first) group of the comparison.
“—” indicates absence of significance.

Comparisons of Equimolar Doses

TABLE 12
Test decisions for equimolar doses - Days 88-82

| | Day | | | | | | |
|---|---|---|---|---|---|---|---|
| Comparison | 88 | 87 | 86 | 85 | 84 | 83 | 82 |
| Day 1, 4, 7 | | | | | | | |
| schedule | | | | | | | |
| 2 vs. 6 | # | # | # | # | # | — | — |
| 3 vs. 7 | # | # | # | # | # | # | # |
| Day 1-7 | | | | | | | |
| schedule | | | | | | | |
| 4 vs. 9 | # | # | # | # | — | — | — |
| 5 vs. 10 | # | # | # | # | # | — | — |
| 5 vs. 11 | # | # | # | # | # | # | — |

note:
* (#) indicates that first (second) group shows a significantly (p < 0.05) larger weight reduction than second (first) group of the comparison.
“—” indicates absence of significance.

TABLE 13
Test decisions for equimolar doses - Days 89-108

| | Day | | | | | | |
|---|---|---|---|---|---|---|---|
| Comparison | 89 | 91 | 94 | 96 | 98 | 101 | 108 |
| Day 1, 4, 7 | | | | | | | |
| schedule | | | | | | | |
| 2 vs. 6 | # | # | # | # | — | — | — |
| 3 vs. 7 | # | # | # | # | # | # | # |
| Day 1, 4, 7 | | | | | | | |
| schedule | | | | | | | |
| 4 vs. 9 | # | # | # | — | — | — | — |
| 5 vs. 10 | # | # | # | # | # | — | — |
| 5 vs. 11 | # | # | # | # | # | # | # |

note:
* (#) indicates that first (second) group shows a significantly (p < 0.05) larger weight reduction than second (first) group of the comparison.
“—” indicates absence of significance.

Comparisons of Treatment Schedules

TABLE 14
Test decisions for comparison of treatment schedules
(Day 1, 4, 7 vs. Day 1-7) - Day 88-82

| | Day | | | | | | |
|---|---|---|---|---|---|---|---|
| Comparison | 88 | 87 | 86 | 85 | 84 | 83 | 82 |
| Non-fused | | | | | | | |
| AXOKINE ® | | | | | | | |
| 2 vs. 4 | # | # | # | # | # | # | — |
| 3 vs. 5 | # | # | # | # | # | # | — |
| C-albumin-fused | | | | | | | |
| AXOKINE ® | | | | | | | |
| 6 vs. 9 | # | # | # | # | # | # | — |
| 7 vs. 10 | # | # | # | # | # | — | — |
| 7 vs. 11 | # | # | # | # | # | # | # |

note:
* (#) indicates that first (second) group shows a significantly (p < 0.05) larger weight reduction than second (first) group of the comparison.
“—” indicates absence of significance.

TABLE 15
Test decisions for comparison of treatment schedules
(Day 1, 4, 7 vs. Day 1-7) - Day 89-108

| | Day | | | | | | |
|---|---|---|---|---|---|---|---|
| Comparison | 89 | 91 | 94 | 96 | 98 | 101 | 108 |
| Non-fused | | | | | | | |
| AXOKINE ® | | | | | | | |
| 2 vs. 4 | # | # | # | # | # | — | — |
| 3 vs. 5 | # | # | # | # | # | # | # |
| C-albumin-fused | | | | | | | |
| AXOKINE ® | | | | | | | |
| 6 vs. 9 | # | # | # | # | # | # | # |
| 7 vs. 10 | # | # | # | # | # | — | — |
| 7 vs. 11 | # | # | # | # | # | # | — |

note:
* (#) indicates that first (second) group shows a significantly (p < 0.05) larger weight reduction than second (first) group of the comparison.
“—” indicates absence of significance.

Potency Estimation

TABLE 16
Potency estimations using Day 88 body weight change^{#}

| Comparison | Potency |
|---|---|
| 7 daily injection: albumin-fused AXOKINE ®/AXOKINE ® | 1.90 |
| Day 1, 4, 7 injection: albumin-fused AXOKINE ®/ | 2.33 |
| AXOKINE ® | |
| albumin-fused AXOKINE ®: 7 daily/Day 1, 4, 7 | 9.13 |
| Non fused AXOKINE ®: 7 daily/Day 1, 4, 7 | 1.85 |
| Day 1, 4, 7 injection albumin-fused AXOKINE ®/7 daily | 0.87 |
| injections non fused AXOKINE ® | |

^{#}Parallel line assay for Day 88 body weight change on log-dose was used. Group 1 and 11 were not included in the calculations.

The observed prolongation of the plasma half life of albumin-fused AXOKINE as investigated in rabbits (72 times longer than non-fused AXOKINE) while administering the fusion protein s.c. is very surprising. First the s.c. administration is known to result in reduced resorption which is not the case here. Second as it is normally known that plasma half lives of human plasma proteins are sometimes dramatically reduced in animals this prolongation points out that the situation in humans is even more pronounced. This is confirmed by our pharmacodynamic findings in mice, where it was possible to administer the fusion protein every third day with nearly comparable efficacy compared to the daily application of the non-fused AXOKINE. As a consequence we speculate that it might be possible to administer the fusion protein perhaps in humans in weekly or even longer intervals. Furthermore efficacy and safety might be increased as could be a decreased rate of the generation of antibodies towards CNTF.

Clinical Observations

Six animals were prematurely withdrawn from the study, all after completion of the treatment:

Starting on day 84 all animals of groups 8, 10 and 11 (receiving 1200 μg/kg C-AFP daily or 3600 μg/kg C-AFP 3×) showed a dull, ruffled coat, generalized reddening of the skin and reduced general condition. Up to 10 of the 12 animals receiving 1200 μg and all animals treated with 3600 μg developed bloody diarrhea over the following two days, accompanied by reduced water intake, leading to severe dehydration.

Therefore one animal in each of the groups 10, 11 on Day 89, three animals in group 8 on Day 91 were killed. One further animal of group 10 died on Day 96.

At necropsy severe obesity, dehydration, and fatty degeneration of liver and kidneys, together with dilated intestines were found in all examined animals.

### CONCLUSION

Prior to start of treatment (Day 81) a total of 70 animals were available, 10 animals in the placebo group (group 1), and six animals in each of the 10 active treatment groups. A total of six animals were killed or died during the study course, all after completion of the treatment: one animal in each of the groups 10, 11 on Day 89, three animals in group 8 on Day 91, and finally one further animal in group 10 on Day 96. These cases and the observed clinical symptoms were confined to the highest dose groups, and are thus considered as treatment-related.

The weight of placebo treated animals was nearly constant between Day 81 and Day 88 (mean weight change on Day 88: −0.4%), but in the further course of the trial a weight increase until Day 108 (mean change on Day 108: 7.2%) was noticed.

Treatment with active substances (groups 2-11) led to significant dose-dependent body weight reductions as compared with placebo (Table 10). Even within 21 days after treatment completion animals treated with an active substance showed significantly higher body weight reductions than placebo animals (Table 11).

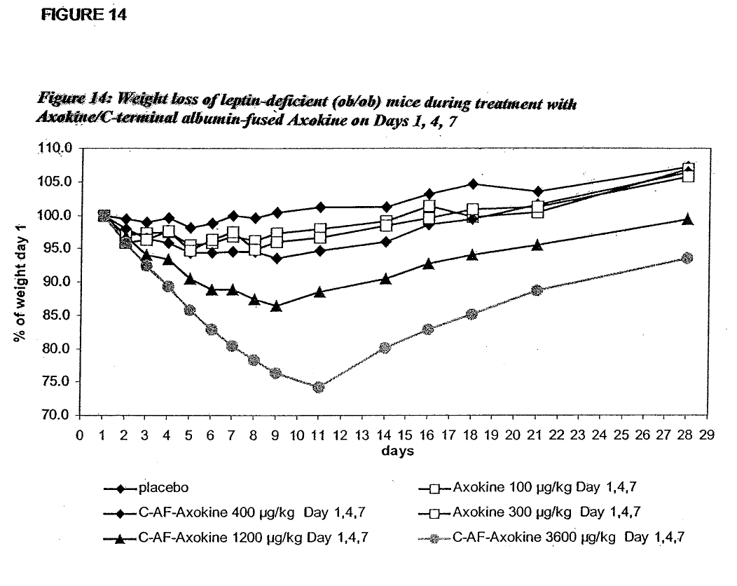

When comparing equimolar doses, albumin-fused AXOKINE® was considerably better than non-fused AXOKINE® with respect to the body weight reduction (Table 12, FIG. 14), no matter which treatment schedule was applied. After the end of treatment this effect continued dose-dependently (Table 13), for groups 7, 11 even until Day 108.

Daily injections over seven days resulted in a more pronounced effect than injections on Days 1, 4, 7 (Table 7, 8), for both under therapy and during the 21 follow-up period. This held for the comparisons within non-fused AXOKINE® and within C-albumin fused AXOKINE.

Potency estimations were confined to the body weight change on Day 88. albumin-fused AXOKINE® was 1.9 and 2.3 times more potent than non-fused AXOKINE® for the seven days treatment schedule and the schedule with treatment on Days 1, 4, 7, respectively (Table 16). Treatments on Days 1-7 were more potent than treatments on Day 1, 4, 7. For non-fused AXOKINE® a potency of 1.85 and for the albumin-fused AXOKINE a potency of 9.13 was calculated.

Injections with albumin-fused AXOKINE® on Day 1, 4, 7 were nearly as potent as daily injections on seven consecutive days with unfused AXOKINE.

### LIST OF CITED REFERENCES

1. Kalra S P, Dube M G, Pu S, Xu B. Horvath T L, Kalra P S. Interacting appetite-regulating pathways in the hypothalamic regulation of body weight. Endocrine Reviews 1999; 20: 68-100.
2. Ahima R S, Osei S Y. Molecular regulation of eating behaviour: new insights and prospects for therapeutic strategies. TRENDS in Molecular Medicine 2001; 7: 205-213.
3. Dove A. Biotech weighs up the options in obesity. Nature Biotechnology 2001; 19: 25-28.
4. Van der Ploeg, LHT. Obesity: an epidemic in need of therapeutics. Current opinion in Chemical Biology 2000; 4: 452-460.
5. Wieland H A, Hamilton B S. Weighing the options in the pharmacotherapy of obesity. International Journal of Clinical Pharmacology and Therapeutics 2001; 39: 406-414.
6. Inui, A. Transgenic study of energy homeostasis equation: implications and confounding influences. The FASEB Journal 2000; 14: 2158-2170.
7. Lambert P D, Anderson K D, Sleeman M W, Wong V, Tan J, Hijarunguru A, Corcoran T L, Murray J D, Thabet K E, Yancopoulos G D, Wiegand S J. Ciliary neurotrophic factor activates leptin-like pathways and reduces body fat, without cachexia or rebound weight gain, even in leptin-resistant obesity. PNAS 2001; 98: 4652-4657.
8. ALS CNTF Treatment Study Group: A double-blind placebo-controlled clinical trial of subcutaneous recombinant human ciliary neurotrophic factor (rHCNTF) in amyotrophic lateral sclerosis. Neurology 1996; 46: 1244-1249.
9. Kalra S P. Circumventing leptin resistance for weight control. PNAS 2001; 98: 4279-4281.
10. Podusio J F, Curran G L. Permeability at the blood-brain and blood-nerve barriers of the neurotrophic factors: NGF, CNTF, NT-3, BDNF. Molecular Brain Research 1996; 36: 280-286.
11. Pan W, Kastin A J, Maness L M, Brennan J M. Saturable entry of ciliary neurotrophic factor into brain. Neuroscience Letters 1999; 263: 69-71.
12. Bickel U, Yoshikawa T, Pardridge W M. Delivery of peptides and proteins through the blood-brain barrier. Advances in Drug Delivery Review 2001; 46: 247-79
13. Gloor S M, Wachtel M, Bolliger M F, Ishihara H, Landmann R, Frei K. Molecular and cellular permeability control at the blood-brain barrier. Brain Research Reviews 2001; 36: 258-264.
14. Guler H P, Acheson A, Stambler N, Hunt T L, Dato M. Safety study with AXOKINE® (Rm): a second generation ciliary neurotrophic factor. International Journal of Obesity and Related Metabolic Disorders. 24 (Suppl. 1): 102, May 2000. USA.
15. Guler H P, Ettinger M P, Littlejohn T W, Schwartz S L, Weiss S L, Mcliwain H H, Heymsfield S B, Bray G A, Roberts W G, Acheson A, Heyman E R, Dark C L, Vicary C. AXOKINE® causes significant weight loss in severely and morbidly obese subjects. International Journal of Obesity and Related Metabolic Disorders 2001; 25: S111: P 291.
16. Regeneron Pharmaceuticals Inc. Regeneron gets positive weight loss results from AXOKINE. Media Release: [6 pages], 29 Nov. 2000. Available from: URL: http://www.regeneron.com. USA.
17. Regeneron Pharmaceuticals Inc. Regeneron updates phase II obesity trial results; weight loss maintained 36 weeks following treatment cessation. Media Release: [6 pages], 11 Sep. 2001. Available from: URL: http://www.regeneron.com. USA.
18. Regeneron Pharmaceuticals Inc. Regeneron updates obesity drug results; patients maintained weight loss following treatment cessation. Media Release: [4 pages], 28 Feb. 2001. Available from: URL: http://www.regeneron.com. USA.
19. SCRIP No 2720 February 2002: Question over AXOKINE® safety.

1. A fusion protein comprising an albumin, or a fragment or a variant or a derivative thereof, and at least one biologically active peptide or protein which activates the ciliary neurotrophic factor (CNTF) receptor, or a fragment or variant or a derivative thereof.

2. The fusion protein of claim 1, wherein the at least one peptide or protein which activates the ciliary neurotrophic factor (CNTF) receptor is CNTF or a fragment or variant or a derivative thereof.

3. The fusion protein of claim 2, wherein the CNTF is AXOKINE®.

4. The fusion protein of claim 1 wherein the in-vivo half-life of the fusion protein is greater than the in-vivo half-life of the unfused biologically active peptide or protein.

5. The fusion protein of claim 1 wherein the shelf-life of the fusion protein is greater than the shelf-life of the unfused biologically active peptide or protein.

6. The fusion protein of claim 1 which is expressed in yeast.

7. The fusion protein of claim 1 which is expressed in a mammalian cell.

8. The fusion protein of claim 1 wherein the mammalian cell is a human cell.

9. A pharmaceutical composition comprising an effective amount of the fusion protein of claim 1 and a pharmaceutically acceptable carrier or excipient.

10. A method of treating obesity and diseases associated therewith comprising administering the fusion protein of claim 1.

11. The method of claim 10, wherein the disease associated with obesity is diabetes, hyperglycaemia or hyperinsulinemia.

12. A method for minimizing side effects associated with the treatment of a mammal with a biologically active peptide or protein activates the ciliary neurotrophic factor (CNTF) receptor, or a fragment or variant or a derivative thereof, the method comprising linking said biologically active peptide or protein to an albumin to form an albumin-fused biologically active peptide or protein and administering said albumin-fused biologically active peptide or protein to said mammal.

13. The method of claim 12, wherein the biologically active peptide or protein activates the ciliary neurotrophic factor (CNTF) receptor, or is a fragment or variant or a derivative thereof.

14. The method of claim 12, wherein the biologically active peptide or protein is CNTF or a fragment or variant or a derivative thereof.

15. The method of claim 12, wherein said side effect is nausea, headache, or a combination of nausea and headache.


SEQUENCE LISTING (3 sequences)

SEQ ID NO: 1 - protein, 783 aa, Artificial Sequence
  FUSION PROTEIN
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGSGGSGGSGGAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHILLLQVAAFAYQIEELMILLEYKIPRNEADGMPINVGDGGLFEKKLWGLKVLQELSQWTVRSIHDLRFISSHQTG

SEQ ID NO: 2 - protein, 791 aa, Artificial Sequence
  FUSION PROTEIN
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKFPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDYKDHDGDYKDHDIDYKDDDDKAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWIVRSIHDLRFISSHQTG

SEQ ID NO: 3 - protein, 783 aa, Artificial Sequence
  FUSION PROTEIN
AFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL